(19) United States
(12) Patent Application Publication
(10) Pub. No.: US 2006/0058614 A1
(43) Pub. Date: Mar. 16, 2006

(54) Tomographic image observation apparatus, endoscopic apparatus, and probe used therefor

(75) Inventor: Kazuhiro Tsujita, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/198,349

(22) Filed: Aug. 8, 2005

(30) Foreign Application Priority Data
Aug. 18, 2004 (JP) 2004-237865

(51) Int. Cl. A61B 5/05

(52) U.S. Cl. 600/407

(57) ABSTRACT

A probe capable of obtaining both optical image information and ultrasonic image information without being affected by radiation noise with a relatively simple structure. The probe includes an insertion part to be inserted into a body of an object and having at least one region for transmitting light and ultrasonic waves; a light propagating path formed of a material having flexibility within the insertion part, for propagating the light; at least one ultrasonic propagating path formed of a material having flexibility within the insertion part, for propagating the ultrasonic waves; and a reflection mirror accommodated within the insertion part, for directing the light outputted from an end surface of the light propagating path outward of the insertion part and directing the ultrasonic waves outputted from an end surface of the at least one ultrasonic propagating path outward of the insertion part.

### BACKGROUND OF THE INVENTION

1. Field of the Invention

The present invention relates to a tomographic image observation apparatus and an endoscopic apparatus used for observation of images within a living body in medical diagnoses, and further relates to a probe used in those apparatuses.

2. Description of a Related Art

In medical fields, diagnoses using tomographic images generated by OCT (optical coherence tomography) technology have been made. The OCT refers to a technology of generating tomographic images on an object to be inspected utilizing low-coherence interference of light based on a principle as below. That is, low-coherence light outputted from a light source such as a laser or SLD (super luminescent diode) is split into signal light and reference light, a frequency of the signal light or reference light is slightly shifted by a piezoelectric element or the like, and the signal light is entered into a scanning region. Then, the signal light is reflected at a predetermined depth of the object to form reflected light, the reflected light and the reference light are combined, and the intensity of interference signal contained in the combined light is measured by heterodyne detection. At that time, a mirror or the like located in the optical path of the reference light is moved to change the optical path length of the reference light, and thereby, information on the object at a depth at which the optical path length of the reference light and the optical path length of the signal light become the same can be obtained. Accordingly, measurement is performed while shifting the irradiated region of the signal light and changing the optical path length of the reference light, and thereby, optical tomographic images on a predetermined region can be obtained. As for details of the OCT, refer to Japanese Patent Application Publication JP-P2002-148185A (page 2).

Since tomographic images with high resolution on the order from 10 μm to 20 μm can be obtained by using such OCT, the OCT is being applied to various fields. For example, EOCT (endoscopic optical coherence tomography) introducing the OCT into an endoscope is reported in Akihiro HORII, “EOCT (Endoscopic Optical Coherence Tomography)”, Journal of the Japan Society for Precision Engineering, Vol. 67, No. 4, 2001, pp. 550-553.

However, the reachable depth of light is as shallow as about 2 mm from the surface of the tissue, and therefore, there has been a problem that image information only on the shallow part of the living tissue can be obtained in the OCT.

On the other hand, as a technology of generating tomographic images of the object, ultrasonic imaging is also known. The ultrasonic imaging is a technology of transmitting ultrasonic waves into the object by using an ultrasonic transducer, receiving ultrasonic waves (ultrasonic echoes) reflected at boundaries of tissues within the object or the like to generate tomographic images based on the reception signals. According to the ultrasonic imaging, assuming that the resolution of the tomographic image is several hundreds of micrometers, since the reachable depth of ultrasonic waves is as deep as about 10 mm, image information on the deep part of the living tissue can be obtained. Accordingly, it is expected that image information in a broader region with respect to the depth direction can be obtained by combining the ultrasonic diagnosis and the above-mentioned OCT.

Japanese Patent Application Publication JP-A-11-56752 discloses an intra-object tomographic imaging apparatus including an insertion probe covered by an outer sheath having an elongated shape and flexibility to be inserted into a body cavity for obtaining three-dimensional image signals by using low coherence light and ultrasonic waves, an optical tomographic image signal detection unit for generating low coherence light, guiding the light to the insertion probe side, with the reflection light from the patient side within the body cavity as light to be measured, detecting the light by allowing the light to interfere with reference light, a signal processing unit for performing signal processing on the interference signal detected by the optical tomographic image signal detection unit or the like and driving an ultrasonic vibrator located on the tip of the insertion probe and performing signal processing on ultrasonic echo signals, and a monitor for displaying a video signal outputted from the signal processing unit (page 1, FIG. 1). Thus, the apparatus has both the function of obtaining OCT signals and the function of transmitting and receiving ultrasonic signals, and thereby, high resolution can be obtained at a depth near the surface of the object and deep tomographic images in the depth direction can be obtained. As a result, appropriate and effective object tomographic observation can be performed.

By the way, in the above-mentioned intra-object tomographic imaging apparatus, an optical fiber and an optical system for the OCT and a substrate, on which a vibrator for generating ultrasonic waves is mounted, are provided on the tip of the probe. However, it is difficult to provide precise and complex parts and mechanisms in such a small region, the cost of manufacturing the probe itself becomes much higher.

Further, in the case where the ultrasonic imaging function is provided to an endoscopic apparatus in which a solid-state image sensor such as a CCD camera is provided within the probe, the noise that the drive signal for generating ultrasonic waves provides to the image signal of the solid-state image sensor becomes problematic. In order to generate ultrasonic waves, a drive signal having a large amplitude equal to or more than several tens of volts having a high frequency within a range from about 7 MHz to about 30 MHz must be transmitted over a probe length within a range from about 2 m to about 3 m, for example. Accordingly, a problem that the radiation noise affects the image signal of the electronic endoscope and deteriorates image quality or the like arises.

As a related technology, a transmission experiment of ultrasonic waves performed for developing a transmission line and an ultrasonic transmission technology capable of transmitting ultrasonic waves within a range from several megahertz to a hundred megahertz with low loss by using extra fine quartz fibers has been reported in Takasuke IRIE et al., “A Transmission Method of 30 MHz (Range) Ultrasonic Wave Using the Fused Quartz Fiber”, the 23rd Symposium on Basics and Application of Ultrasonic Electronics, November, 2002, pp. 3-4. In the above document of IRIE at el., it has been confirmed that high-frequency ultrasonic waves having a frequency range to 50 MHz can be transmitted through the quartz fiber. However, any form to which such an ultrasonic transmission technology is applied is not mentioned.

### SUMMARY OF THE INVENTION

The present invention is achieved in view of the above-mentioned problems. An object of the present invention is to provide a probe capable of obtaining both optical image information and ultrasonic image information without being affected by radiation noise with a relatively simple structure. Another object of the present invention is to provide a tomographic image observation apparatus and an endoscopic apparatus using such a probe.

In order to solve the above-mentioned problems, a probe according to one aspect of the present invention is a probe to be used in optical coherence tomography for generating an image based on interference of low-coherence light and ultrasonic imaging for generating an image based on ultrasonic echoes, and the probe comprises: an insertion part to be inserted into a body of an object to be inspected and having at least one region for transmitting light and ultrasonic waves; light propagating means formed of a material having flexibility and accommodated within the insertion part, the light propagating means having two end surfaces for entering and/or outputting light, and propagating the light entering from one end surface to the other end surface; at least one piece of ultrasonic propagating means formed of a material having flexibility and accommodated within the insertion part, the ultrasonic propagating means having two end surfaces for entering and/or outputting ultrasonic waves, and propagating the ultrasonic waves entering from one end surface to the other end surface; and guide means accommodated within the insertion part, for directing the light outputted from the end surface of the light propagating means outward of the insertion part and directing the ultrasonic waves outputted from the other end surface of the at least one piece of ultrasonic propagating means outward of the insertion part.

Further, an apparatus according to one aspect of the present invention is an apparatus to be used in optical coherence tomography for generating an image based on interference of low-coherence light and ultrasonic imaging for generating an image based on ultrasonic echoes, and the apparatus comprises: light splitting means for splitting light generated from a light source into signal light and reference light; at least one ultrasonic transducer for generating ultrasonic waves based on a drive signal; drive signal generating means for generating the drive signal to be supplied to the at least one ultrasonic transducer; a probe including an insertion part to be inserted into a body of an object to be inspected and having at least one region for transmitting light and ultrasonic waves, light propagating means formed of a material having flexibility and accommodated within the insertion part, for entering the signal light split by the splitting means and propagating the signal light, at least one piece of ultrasonic propagating means formed of a material having flexibility and accommodated within the insertion part, for propagating the ultrasonic waves entering from the at least one ultrasonic transducer, and guide means accommodated within the insertion part, for directing the light outputted from the light propagating means outward of the insertion part and directing the ultrasonic waves outputted from the at least one piece of ultrasonic propagating means outward of the insertion part; detecting means for detecting interference light generated by interference between the signal light reflected from the object and propagated in the light propagating means and the reference light to generate a detection signal; first image data generating means for generating tomographic image data based on the detection signal generated by the detecting means; and second image data generating means for generating tomographic image data based on a detection signal generated by receiving the ultrasonic waves reflected from the object.

According to the present invention, since the ultrasonic waves generated outside of the probe is propagated to the tip of the probe via an ultrasonic wave propagation path having flexibility, there is no need to provide a vibrator within the probe. Further, since there is no need to transmit a high-frequency signal for driving a vibrator to the probe, measures for radiation noise becomes unnecessary. Accordingly, the structure of the probe can be simplified and the diameter thereof can be made smaller, and the cost for manufacturing the probe can be reduced while maintaining image quality of the images to be generated. Furthermore, by incorporating such a probe in a tomographic image observation apparatus or an endoscopic apparatus, a tomographic image obtained by using the ultrasonic waves and a tomographic image or an interior surface image obtained by using the light can be simultaneously displayed, and therefore, medical diagnoses can be performed efficiently.

### BRIEF DESCRIPTION OF THE DRAWINGS

FIG. 1 is a block diagram showing a constitution of a tomographic image observation apparatus according to one embodiment of the present invention;

FIG. 2 is a sectional view showing a structure of a tomographic image observation probe shown in FIG. 1;

FIGS. 3A and 3B are diagrams for explanation of arrangement of light propagation path and ultrasonic wave propagation path in a bundle fiber shown in FIG. 2;

FIG. 4 is a schematic diagram for explanation of a constitution of a light source unit to a photodetecting unit shown in FIG. 1;

FIG. 5 is a schematic diagram showing a state in which the ultrasonic waves generated from an ultrasonic transducer shown in FIG. 1 are introduced into the ultrasonic wave propagation path;

FIG. 6 is a schematic diagram showing a tomographic image displayed on a display unit shown in FIG. 1;

FIG. 7 is a block diagram showing a constitution of an endoscopic apparatus according to one embodiment of the present invention;

FIG. 8 shows an overview of a part of the endoscopic apparatus shown in FIG. 7;

FIGS. 9A and 9B show the tip portion of the insertion part of the endoscopic probe shown in FIG. 8; and

FIG. 10 shows a state in which the endoscopic probe shown in FIG. 8 is inserted into a gastrointestinal tract of a patient and the OCT and ultrasonic imaging and endoscopic examination are performed.

### DESCRIPTION OF THE PREFERRED EMBODIMENTS

Hereinafter, preferred embodiments of the present invention will be described in detail by referring to the drawings. The same reference number will be assigned to the same component and the description thereof will be omitted.

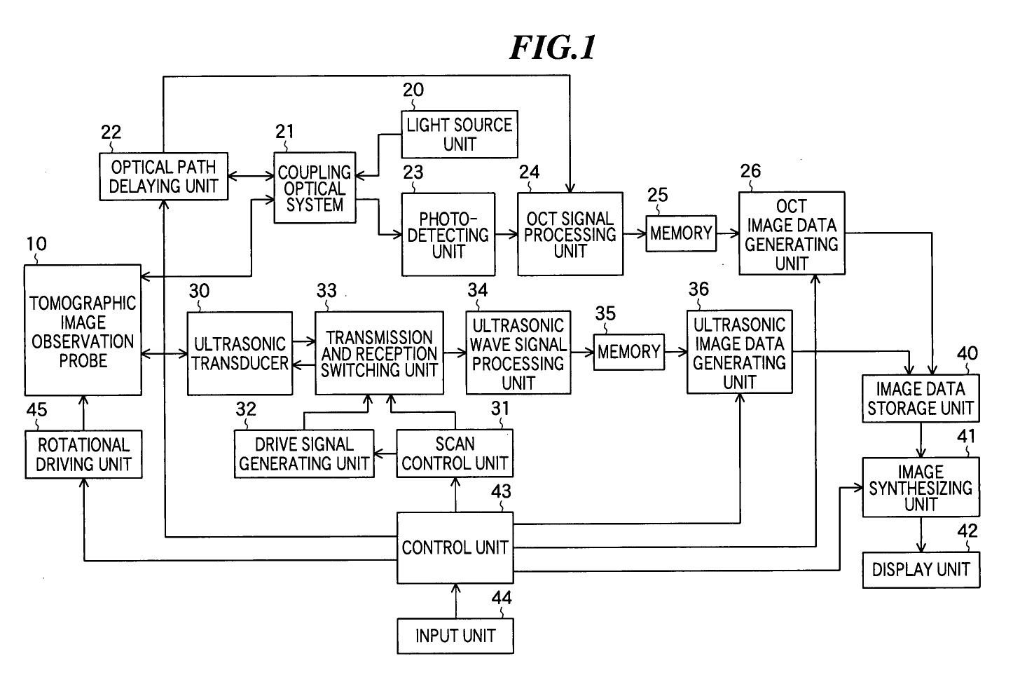

FIG. 1 is a block diagram showing a constitution of a tomographic image observation apparatus according to one embodiment of the present invention. This tomographic image observation apparatus includes a tomographic image observation probe 10 (hereinafter, also simply referred to as “probe”) to be inserted into a living body for OCT (optical coherence tomography) imaging and ultrasonic imaging, a light source unit 20 to an OCT image data generating unit 26 for generating tomographic images by the OCT, an ultrasonic transducer 30 to an ultrasonic image data generating unit 36 for generating tomographic images by using ultrasonic waves, an image data storage unit 40 for storing the generated OCT image data and ultrasonic image data, an image synthesizing unit 41, a display unit 42, a control unit 43 for controlling the entire tomographic image observation apparatus according to the embodiment, and an input unit 44 to be used when an operator inputs instructions and information. Further, a rotational driving unit 45 to be coupled to the probe 10 is provided.

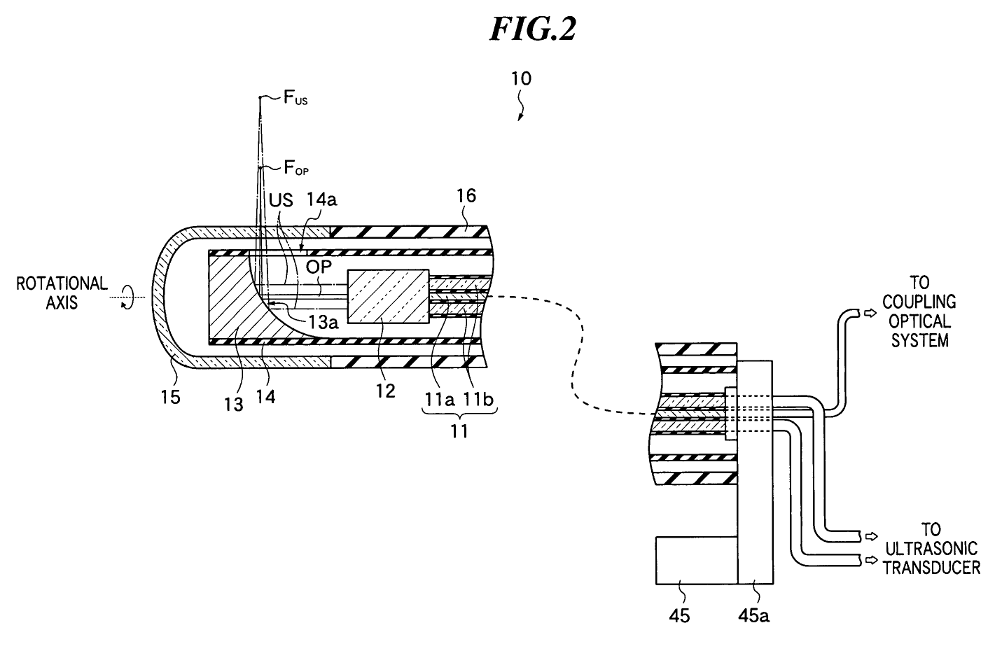

FIG. 2 is a sectional view showing a structure of the probe 10 shown in FIG. 1. This probe 10 includes a bundle fiber 11, a collimator 12, and a reflection mirror 13 rotating around a rotational axis. The bundle fiber 11 and the collimator 12 are inserted into a cladding tube 14 formed of a material having flexibility and secured, and the reflection mirror 13 is attached on the tip of the cladding tube 14. These parts 11 to 14 are accommodated within an insertion part including a soft member 16 to which an end cap 15 is provided. The end cap 15 has optical transparency like glass or resin material, and is formed of a material with good acoustic characteristics to the living body. The space inside of the end cap 15 is filed with a liquid such as water or liquid paraffin. Further, at the other end of the cladding tube 14 (at the right end in FIG. 2), the rotational driving unit 45 such as a motor including a gear portion 45a is provided. The cladding tube 14 is rotated by the rotational driving unit 45, and thereby, the reflection mirror 13 is rotated.

The bundle fiber 11 includes a light propagation path 11a for propagating light used for the OCT and an ultrasonic wave propagation path 11b for propagating ultrasonic waves to be used for ultrasonic imaging. The light propagation path 11a and the ultrasonic wave propagation path 11b are formed of a material having flexibility. As the light propagation path 11a, for example, a single-mode fiber having a core diameter of 10 μm is used, and, as the ultrasonic wave propagation path 11b, for example, a quartz fiber is used. Note that the ultrasonic wave propagation path 11b is not necessarily in a single mode.

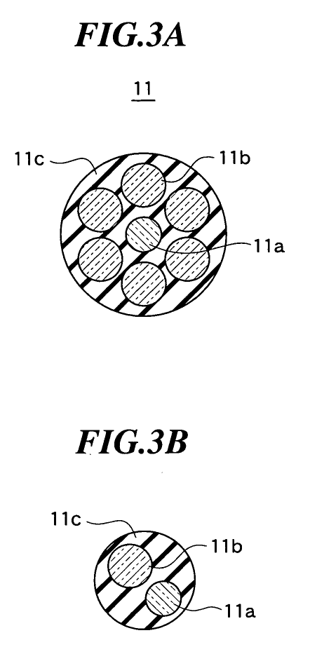

FIGS. 3A and 3B show sections of the bundle fiber 11 shown in FIG. 2. In the embodiment, as shown in FIG. 3A, the light propagation path 11a is located at the center of the bundle fiber 11 and plural ultrasonic wave propagation paths 11b are arranged so as to surround the path 11a. In order to protect the propagation paths 11a and 11b and absorb unwanted vibration, the gap between the paths is filled with a resin material 11c. The arrangement of the light propagation path 11a and the ultrasonic wave propagation path 11b is not limited to the form, but various other arrangements may be used. For example, as shown in FIG. 3B, one light propagation path 11a and one ultrasonic wave propagation path 11b may be located side by side.

Referring to FIG. 2 again, one ends of the light propagation path 11a and the ultrasonic wave propagation path 11b are directly connected to the collimator 12. Further, the other end of the light propagation path 11a is connected to a coupling optical system 21 shown in FIG. 1, and the other end of the ultrasonic wave propagation path 11b is connected to the ultrasonic transducer 30 shown in FIG. 1.

The collimator 12 has a larger aperture diameter than that of the bundle fiber 11. It shapes the wavefront of the light outputted from the end surface of the light propagation path 11a so that the outputted light enters the reflection mirror 13 without being diffused, and propagates the ultrasonic waves outputted from the end surface of the ultrasonic wave propagation path 11b. In the embodiment, a SELFOC (registered trademark) lens is used as the collimator 12. The SELFOC (registered trademark) lens is a refractive index profile lens having different refractive indices according to positions and the optical characteristics vary by changing the length thereof. For example, when the SELFOC (registered trademark) lens is one-quarter of a distance between object and image plane (a pitch at which light is erectedly imaged), the incident light is outputted in a parallel light. By the way, using an imaging optical system such as a convex lens in place of the collimator 12, the light outputted from the light propagation path 11a may be entered into the reflection mirror 13 while the diameter thereof is narrowed.

The reflection mirror 13 has a metal reflection surface 13a, deflects the wavefront of the light OP and the ultrasonic waves US outputted from the collimator 12 to focus them in a predetermined position. The shape of the reflection surface 13a is defined according to the state of the incident light (e.g., parallel light, focused light, or the like), the relationship between the aperture diameter and the position of the focal point F_{OP} of light, the relationship between the aperture diameter of incident ultrasonic waves and the position of the focal point F_{US} of ultrasonic waves, or the like. Simultaneously, the focal length of light and the focal length of ultrasonic waves are set within depth ranges as targets of observation according to properties (e.g., invasion depth) of light and ultrasonic waves, respectively. Since, normally, the shallow part of the object is imaged by the OCT and the deep part of the object is imaged by the ultrasonic waves, the focal length of ultrasonic waves becomes longer than the focal length of light. As a shape of the reflection surface 13a, various shapes such as a flat surface, paraboloidal surface, ellipsoidal surface may be used.

A window 14a for transmitting the light OP and the ultrasonic waves US reflected from the reflection surface 13a is provided in a part of the cladding tube 14. The light and the ultrasonic waves that have been reflected by the reflection mirror 13 are transmitted through the window 14a and the end cap 15 and propagated within the object, and form the focal point F_{OP} of light and the focal point F_{US} of ultrasonic waves. By rotating the cladding tube 14, the reflection mirror 13 rotates and the focal point F_{OP} of light and the focal point F_{US} of ultrasonic waves move within a plane orthogonal to the rotational axis, and thereby, scan the object. Alternatively, by driving the cladding tube 14 slidingly within the soft member, the focal point F_{OP} of light and the focal point F_{US} of ultrasonic waves may be moved to linearly scan the object. Furthermore, three-dimensional scan may be performed by combining rotational movement and sliding movement.

Referring to FIG. 1 again, the tomographic image observation apparatus according to the embodiment has the light source unit 20, the coupling optical system 21, an optical path delaying unit 22, a photodetecting unit 23, an OCT signal processing unit 24, a memory 25, and the OCT image data generating unit 26.

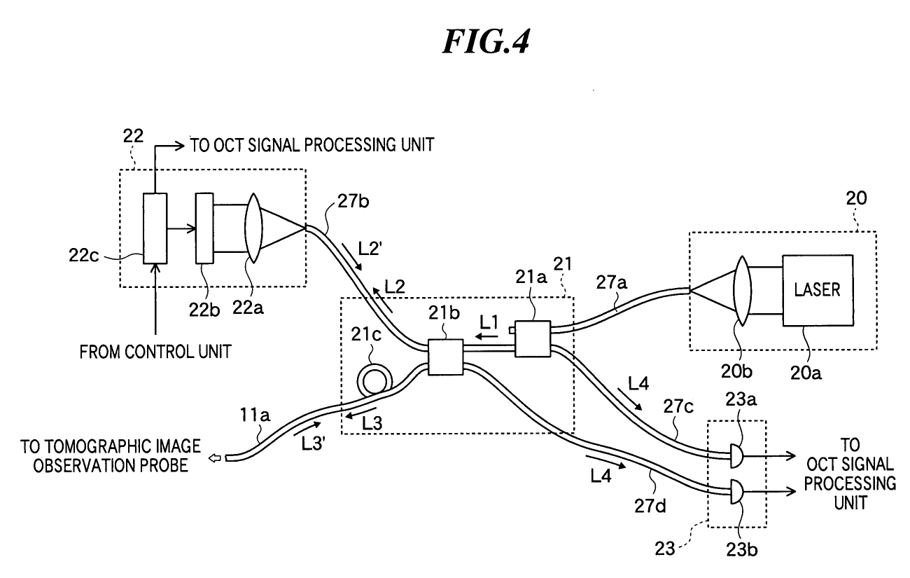

FIG. 4 is a schematic diagram showing a constitution of the light source unit 20 to the photodetecting unit 23. As shown in FIG. 4, the light source unit 20 includes a mode-locked Ti-sapphire laser 20a and a lens 20b for collecting light outputted from the laser 20a and guiding the light to an optical fiber 27a. As the light source, one that can output low-coherence light may be used, and not only the above-mentioned laser, but also an SLD (super luminescent diode) or the like may be used.

The coupling optical system 21 includes fiber couplers 21a and 21b and a frequency shifter 21c. The fiber coupler 21a guides low-coherence light outputted from the light source unit 20 and introduced via the optical fiber 27a to the fiber coupler 21b. The fiber coupler 21b splits low-coherence light L1 into reference light L2 and signal light L3 and guides them to optical fibers 27b and 11a, respectively, and combines reference light L2′ and the reflection light L3′ respectively introduced from the optical fibers 27b and 11a. Then, the fiber coupler 21b splits the combined light L4 again, and introduces one piece of the combined light L4 into an optical fiber 27c via the fiber coupler 21a and the other piece of the combined light L4 into an optical fiber 27d. The frequency shifter 21c slightly frequency-modulates the signal light L3 to generate a slight frequency difference Δf between the reference light L2 and the signal light L3.

The optical path delaying unit 22 includes a lens 22a, a reflection mirror 22b, and a mirror driving unit 22c. The lens 22a collects the reference light L2 outputted from the fiber 27b and enters the light into the reflection mirror 22b, and enters the reflection light (reference light L2′) from the reflection mirror 22b into the optical fiber 27b. Here, the reflection mirror 22b is held orthogonal to the optical axis of the lens 22a and movable in a horizontal direction. The mirror driving unit 22c changes the optical path lengths of the reference lights L2 and L2′ by moving the reflection mirror 22b in the horizontal direction relative to the optical axis under the control of the control unit 43 (FIG. 1).

The photodetecting unit 23 includes a photodetector 23a for detecting the intensity of the combined light L4 that has entered via the optical fiber 27c and a photodetector 23b for detecting the intensity of the combined light L4 that has entered via the optical fiber 27d. The detection signals of the photodetectors 23a and 23b are outputted to the OCT signal processing unit 24 (FIG. 1).

The signal light L3 outputted from the light source unit 20 and entered into the optical fiber 11a via the coupling optical system 21 outputs from the tip of the probe 10 shown in FIG. 2 and illuminates the scanning region of the objects. The signal light L3 is reflected by a tissue at a certain depth within the object, and enters the tip of the probe 10 as the reference light L3′. Then, the reflection light L3′ passes through the optical fiber 11a and enters the coupling optical system 21 again, and is combined with the reference light L2′. Here, the reference light L2′ and the reference light L3′ interfere each other in the case where the difference between the optical path length of the reference light L2 from being reflected at the optical path delaying unit 22 and to returning and the optical path length of the signal light L3 from being reflected at the object and to returning is equal to or less than the interference distance of light (e.g., 10 μm to 20 μm). In other words, when the reference lights L2 and L2′ and the reflection light L3′ interfere, the reflection light L3′ has been reflected at a depth corresponding to the optical lengths of the reference lights L2 and L2′, and the reflection light L3′ is thought to indicate information on the depth region. Accordingly, measuring the interference between the reference light L2′ and the reflection light L3′, i.e., a beat signal in a cycle of the frequency difference Δf between both lights appearing in the combined light L4 while varying the optical lengths of the reference lights L2 and L2′, the information on the depth direction of the object can be obtained.

The OCT signal processing unit 24 shown in FIG. 1 generates OCT detection data based on a detection signal representing the intensity of the combined light L4 outputted from the photodetector 23a and a detection signal representing the intensity of the combined light L4 outputted from the photodetector 23b. The OCT signal processing unit 24 has a differential amplifier. The unit adjusts the input balance between the output value of the photodetector 23a and the output value of the photodetector 23b, amplifies the difference, and cancels noise components and drift components between them. Thereby, a beat signal component is extracted. Furthermore, the OCT signal processing unit 24 A/D converts the amplified signal. Thus generated OCT detection data is associated with the optical lengths of the reference lights L2 and L2′ (relating to the depth at which the signal light L3 is reflected) corresponding to the amount of movement of the reflection mirror 22b in the optical path delaying unit 22 and stored in the memory 25.

The OCT image data generating unit 26 generates OCT image data for display by performing coordinate transformation corresponding to the scanning method (e.g. radial scan) by the probe 10 based on the OCT detection data that has been stored in the memory 25. The generated OCT image data is stored in an image data storage unit 40.

On the other hand, in order to generate ultrasonic images, the tomographic image observation apparatus according to the embodiment has the ultrasonic transducer 30, a scan control unit 31, a drive signal generating unit 32, a transmission and reception switching unit 33, an ultrasonic wave signal processing unit 34, a memory 35, and the ultrasonic image data generating unit 36.

The ultrasonic transducer 30 is fabricated by a vibrator with electrodes formed on both ends of a material having a piezoelectric property (piezoelectric material) such as a piezoelectric ceramic represented by PZT (Pb (lead) zirconate titanate) or a polymeric piezoelectric element represented by PVDF (polyvinylidene difluoride). When a voltage is applied to such a vibrator by sending a pulsing electric signal or a continuous wave electric signal, the piezoelectric material expands and contracts. By the expansion and contraction, pulsing ultrasonic waves or continuous ultrasonic waves are generated from the vibrator. Further, the vibrator expands and contracts by receiving the propagating ultrasonic waves and generate an electric signal. The electric signal is outputted as a detection signal of the ultrasonic waves.

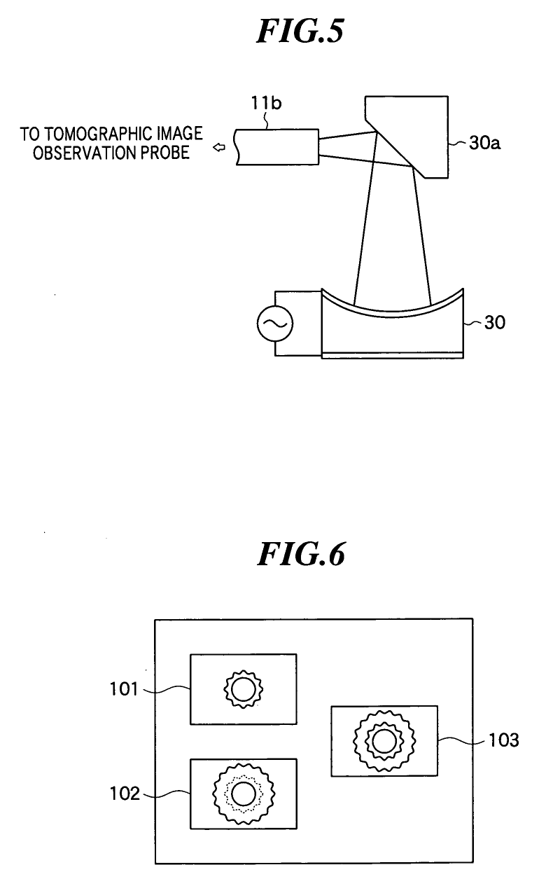

FIG. 5 is a schematic diagram showing a state in which the ultrasonic waves generated from the ultrasonic transducer 30 are introduced into the ultrasonic wave propagation path 11b extending from the probe 10. The ultrasonic transducer 30 has a convex ultrasonic wave generation surface for focusing the generated ultrasonic waves. The ultrasonic waves generated by applying a voltage to such an ultrasonic transducer 30 are reflected by an acoustic mirror 30a and enters the ultrasonic wave propagation path 11b. The reflection surface of the acoustic mirror 30a may be a flat surface as shown in FIG. 5 or concave surface.

The same number of such ultrasonic transducers 30 may be provided as the number of ultrasonic wave propagation paths 11b included in the probe 10, or plural kinds of ultrasonic transducers 30 having different resonance frequencies may be prepared for one ultrasonic wave propagation path 11b. In the latter case, the kinds of ultrasonic transducers 30 to be used may be switched according to a condition of the depth or property of the imaging part. For example, in the case of imaging a relatively shallow region, a transducer for generating ultrasonic waves in a high frequency band, with which high resolution can be obtained, may be used. In the case of imaging a relatively deep region, a transducer generating ultrasonic waves in a low frequency band, which is hard to be diffused and has a deep invasion depth, can be obtained may be used.

Referring to FIG. 1 again, the scan control unit 31 sets driving timing of the drive signal to be provided to the ultrasonic transducer in accordance with the rotational movement of the probe 10 and under control of the control unit 43. Further, the drive signal generating unit 32 includes a pulser, for example, and generates a drive signal according to the driving timing set by the scan control unit 31.

The transmission and reception switching unit 33 switches the supply of the drive signal outputted from the drive signal generating unit 32 to the ultrasonic transducer 30 and the supply of the detection signal outputted from the ultrasonic transducer 30 to the ultrasonic wave signal processing unit 34 with predetermined timing according to the control of the scan control unit 31.

The ultrasonic wave signal processing unit 34 has plural channels corresponding to the number of ultrasonic wave propagation paths 11b. The unit loads the detection signal outputted from the corresponding ultrasonic transducer with predetermined timing, performs signal processing such as logarithmic amplification, detection, STC (sensitivity time control), filter processing, etc., and further performs A/D conversion to generate ultrasonic detection data. Here, by limiting the time period for loading detection signals, ultrasonic echo signals reflected from a specific depth of the object are detected. Thus, generated ultrasonic detection data is stored in the memory 35.

The ultrasonic image data generating unit 36 generates ultrasonic image data for display by performing coordinate transformation corresponding to the scanning method by the probe 10 based on the ultrasonic detection data that has been stored in the memory 35. The generated ultrasonic image data is stored in the image data storage unit 40.

The image synthesizing unit 41 generates synthesized image data for screen display based on the OCT image data and the ultrasonic image data that have been stored in the image data storage unit 40. As a synthesizing method of images, for example, it is conceivable that the OCT image data representing a shallower region than the predetermined depth and the ultrasonic image data representing a deeper region than the predetermined depth are synthesized. An image processing unit for performing tone correction etc. may be provided in the preceding or subsequent stage of the image synthesizing unit 41.

The display unit 42 is a display device including a CRT display or LCD display, and displays images generated by OCT imaging and ultrasonic imaging based on the synthesized image data for screen display that has generated by the image synthesizing unit.

FIG. 6 is a schematic diagram showing a screen displayed on the display unit 42. In FIG. 6, an OCT image 101 in which a shallow part of the imaging region is clearly shown, an ultrasonic image 102 in which a deep part of the imaging region is shown, and a synthesized image 103 generated by synthesizing the shallow part in the OCT image and the deep part in the ultrasonic image are shown. An operator can display each of the OCT image 101, the ultrasonic image 102, and the synthesized image 103 singly, or plural images in a layout as shown in FIG. 6 by inputting instructions using the input unit 45.

As described above, according to the embodiment, a good-quality tomographic image from the shallow part to the deep part can be obtained by one scan by using a probe capable of the OCT and ultrasonic imaging. Accordingly, high quality medical diagnoses can be performed efficiently using such tomographic images. Here, since the ultrasonic waves generated outside of the probe are propagated to the tip of the probe, the structure of the probe itself can be simplified and the diameter thereof can be made smaller. Therefore, the reduction in probe diameter and imaging period can reduce the burden on the patient as an object to be inspected.

Further, according to the embodiment, since the use of plural kinds of ultrasonic transducers having different resonance frequencies can be switched, ultrasonic waves in various frequency bands can be appropriately used according to the imaging parts. In addition, since the restriction on the size of the ultrasonic transducer is reduced, an inexpensive and large ultrasonic transducer can be used and the cost for manufacturing can be reduced.

Furthermore, according to the embodiment, since one reflection mirror capable of reflecting light and ultrasonic waves is used in the probe, the light and the ultrasonic waves can be outputted in the same rotational direction. Accordingly, information on a shallow part and a deep part with respect to a certain region can be simultaneously obtained, and thereby, good-quality images with little time lag in the depth direction can be generated.

In the embodiment, time domain OCT for measuring time change of the interference signal is used, however, also spectrum domain OCT for measuring frequency response characteristic of the interference signal or Fourier domain OCT may be used.

Further, in the embodiment, the ultrasonic echoes are received by using the ultrasonic transducer that has transmitted the ultrasonic waves, however, a transducer for ultrasonic transmission and a transducer for ultrasonic reception may be appropriately used. In this case, since it is unnecessary to supply the drive signal to the transducer for ultrasonic reception, the transducer for ultrasonic reception can be provided on the tip of the probe. Thereby, since the received ultrasonic echo is converted into an electric signal without attenuation while propagation for a long distance, the S/N ratio can be improved.

Next, an endoscopic apparatus according to one embodiment of the present invention will be described. The endoscopic apparatus can not only the OCT and ultrasonic imaging but also endoscopic observation, however, the OCT function may be omitted and only the ultrasonic imaging and endoscopic observation may be performed.

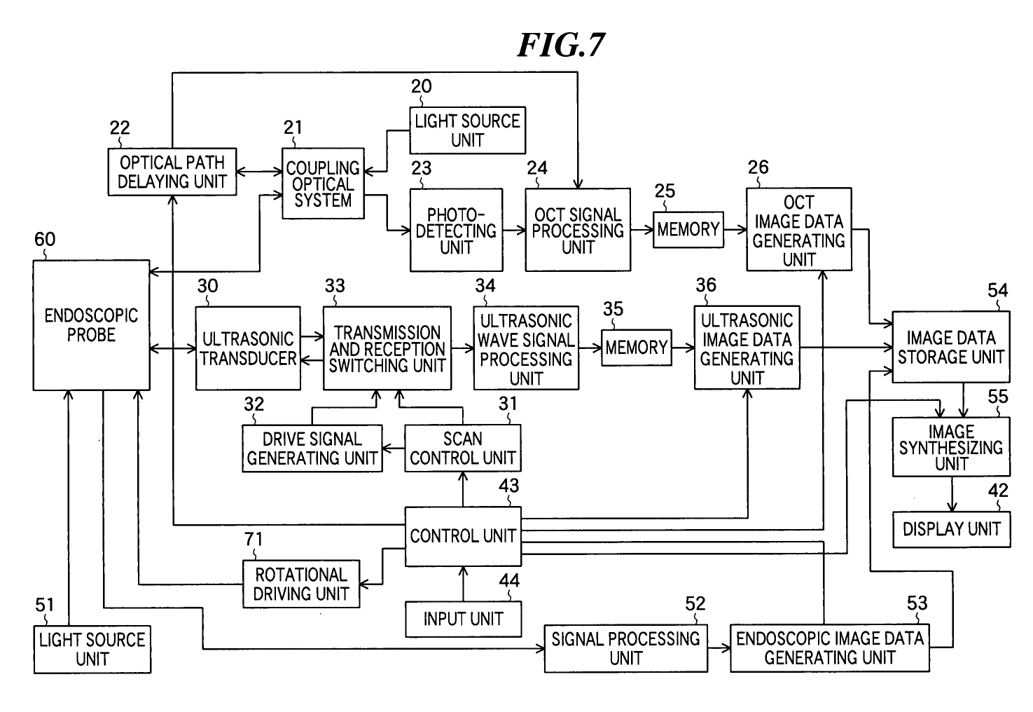

FIG. 7 is a block diagram showing a constitution of an endoscopic apparatus according to the embodiment. The endoscopic apparatus has an endoscopic probe 60 and a rotational driving unit 71 in place of the tomographic image observation probe 10 and the rotational driving unit 45 shown in FIG. 1, and has an image data synthesizing unit 54 and an image synthesizing unit 55 in place of the image data storage unit 40 and the image synthesizing unit 41. Further, the endoscopic apparatus has a light source unit 51, a signal processing unit 52, and an endoscopic image data generating unit 53.

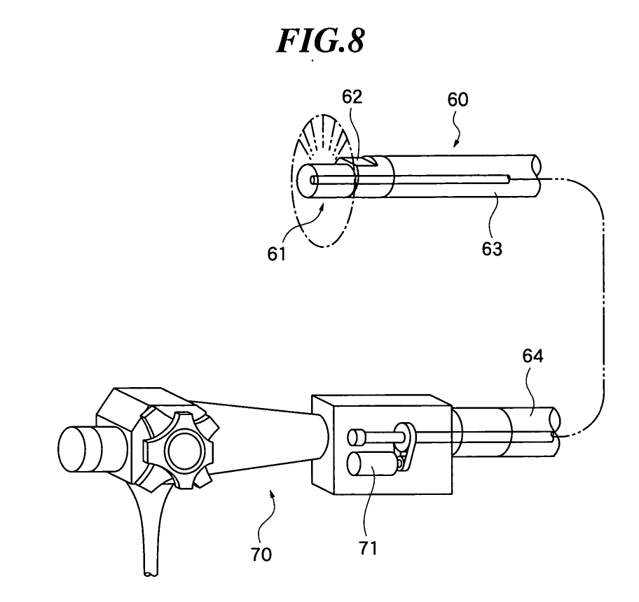

FIG. 8 is a schematic diagram showing an overview of a part of the endoscopic apparatus shown in FIG. 7. The endoscopic apparatus includes the endoscopic probe 60 to be inserted into a body cavity of a patient as an object to be inspected, and a main body operation unit 70 installed in a predetermined location and used for operating the endoscopic probe 60.

In the insertion part of the endoscopic probe 60, an OCT and ultrasonic observation portion 61 and an endoscopic observation portion 62 are provided. Further, the insertion part of the endoscopic probe 60 includes an angle portion 63 and a soft portion 64 and used with the soft portion 64 connected to the main body operation unit 70. Furthermore, the main body operation unit 70 includes the rotational driving unit 71 such as a motor.

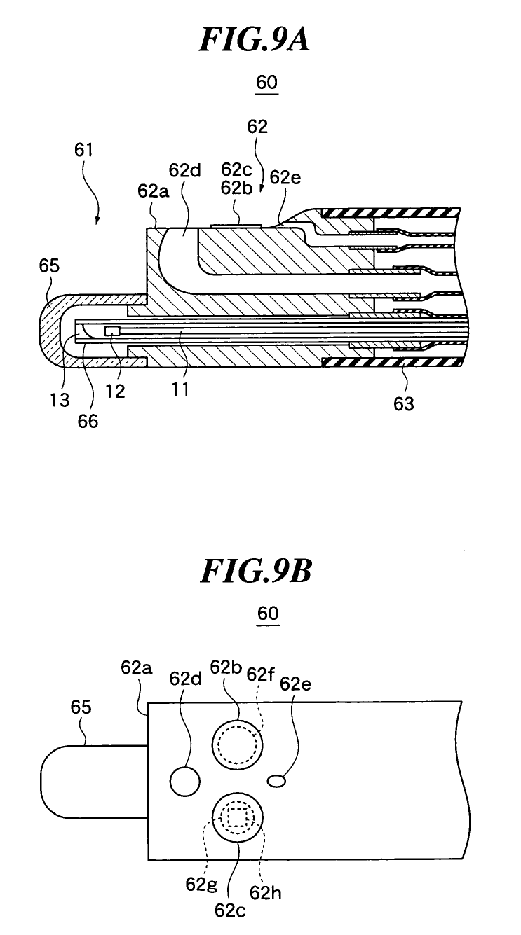

FIG. 9A is a sectional view showing the tip portion of the insertion part of the endoscopic probe shown in FIG. 8. The OCT and ultrasonic observation portion 61 has an end cap 65 projecting from the insertion part and a cladding tube 66 connected to the rotational driving unit 71 shown in FIG. 8 is provided within the insertion part. Inside of the cladding tube 66, similarly to the probe 10 shown in FIG. 2, a bundle fiber 11, a reflection mirror 12, and a collimator 13 are provided. The end cap 65 is filled with a liquid.

FIG. 9B is a top view showing the tip of the insertion part of the endoscopic probe shown in FIG. 8. The endoscopic observation portion 62 has an illumination window 62b and an observation window 62c provided in an observation mechanism mounting portion flattened by chamfering a part of the side surface of the insertion part. An illumination lens 62f for outputting illumination light supplied via a light guide from the light source unit 51 (FIG. 7) for illuminating the interior surface of the object is attached to the illumination window 62b. Further, an objective lens 62g is attached to the observation window 62c, and, in a position where the objective lens forms an image, an input end of an image guide or solid-state image sensor 62h such as a CCD camera is disposed.

Furthermore, a treatment tool lead-out hole 62d for leading out a treatment tool such as forceps is formed in front of the observation window 62c. Further, a nozzle hole 62e for supplying a liquid for cleansing the illumination window 62b and the observation window 62c at the step region of the chamfered part.

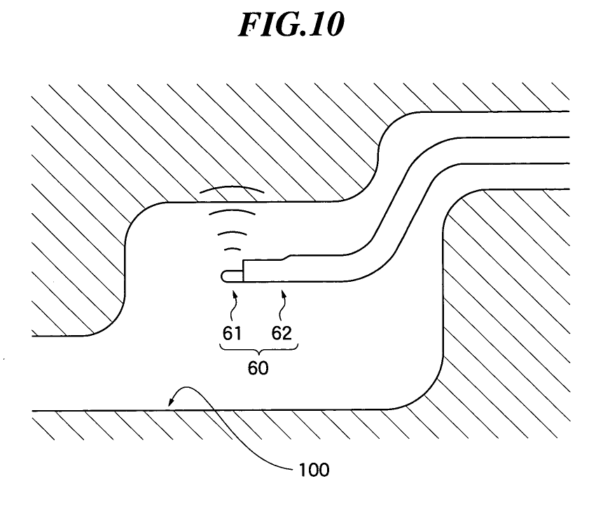

As shown in FIG. 10, the endoscopic probe 60 including the ultrasonic observation portion 61 and the endoscopic observation portion 62 is inserted into a gastrointestinal tract 100 of the patient as the object and the OCT and ultrasonic imaging and endoscopic examination are performed.

Referring to FIG. 7 again, the light generated from the light source 51 is guided to the endoscopic probe 60 and used for illuminating the interior of the object. As the light source 51, for example, a halogen light source or xenon light source is used. The signal processing unit 52 performs predetermined signal processing on the detection signal outputted from the solid-state image sensor provided within the observation window 62c. The endoscopic image data generating unit 54 generates image data representing surface image (endoscopic image) within the object based on the detection signal that has been subjected to signal processing. The image data storage unit 54 stores image data respectively generated by the OCT image data generating unit 26, the ultrasonic image data generating unit 36, and the endoscopic image data generating unit 53. The image synthesizing unit 55 synthesizes tomographic image data based on the OCT image data and the ultrasonic image data that have been stored in the image data storage unit 54, and generates synthesized image data for screen display based on the synthesized tomographic image data and the endoscopic image data. As a display method of screen, each of the OCT image, the ultrasonic image, the synthesized tomographic image and the endoscopic image may be displayed singly and sequentially, or plural images or all images of them may be displayed simultaneously in a layout. An image processing unit for performing tone correction etc. may be provided in the preceding or subsequent stage of the image synthesizing unit 41.

As described above, according to the embodiment, the tomographic image obtained by the OCT and ultrasonic imaging and the surface images of the interior of the living body obtained by endoscopic imaging are obtained by one examination. Accordingly, high quality medical diagnoses can be performed efficiently using those images and the burden on the patient can be reduced. Further, in the case where the ultrasonic transducer is provided at the tip of the probe, because measures for noise etc. that has been essential to the drive signal to be transmitted is not required, the structure of the probe can be simplified.

1. A probe to be used in optical coherence tomography for generating an image based on interference of low-coherence light and ultrasonic imaging for generating an image based on ultrasonic echoes, said probe comprising:
an insertion part to be inserted into a body of an object to be inspected and having at least one region for transmitting light and ultrasonic waves;
light propagating means formed of a material having flexibility and accommodated within said insertion part, said light propagating means having two end surfaces for entering and/or outputting light, and propagating the light entering from one end surface to other end surface;
at least one piece of ultrasonic propagating means formed of a material having flexibility and accommodated within said insertion part, said ultrasonic propagating means having two end surfaces for entering and/or outputting ultrasonic waves, and propagating the ultrasonic waves entering from one end surface to other end surface; and
guide means accommodated within said insertion part, for directing the light outputted from the other end surface of the light propagating means outward of said insertion part and directing the ultrasonic waves outputted from the other end surface of said at least one piece of ultrasonic propagating means outward of said insertion part.

2. A probe according to claim 1, wherein said guide means includes collimating means for shaping wavefront of the light outputted from the other end surface of said light propagating means and propagating the ultrasonic waves outputted from the other end surface of said ultrasonic propagating means.

3. A probe according to claim 1, wherein said guide means includes reflecting means for reflecting the light outputted from the other end surface of said light propagating means outward of said insertion part, and reflecting the ultrasonic waves outputted from the other end surface of said at least one piece of ultrasonic propagating means outward of said insertion part.

4. A probe according to claim 3, wherein said reflecting means reflects the light outputted from the other end surface of said light propagating means such that the light forms a focal point at a predetermined depth, and reflects the ultrasonic waves outputted from the other end surface of said ultrasonic propagating means such that the ultrasonic waves forms a focal point at a predetermined depth.

5. A probe according to claim 3, wherein said guide means further includes a rotation mechanism for rotating said reflecting means to change a direction in which the light and the ultrasonic waves are reflected.

6. A probe according to claim 1, wherein:
said light propagating means includes an optical fiber; and
said at least one piece of ultrasonic propagating means includes plural quartz fibers.

7. A probe to be used in at least endoscopic imaging and ultrasonic imaging, said probe comprising:
an insertion part to be inserted into a body of an object to be inspected and having at least one region for transmitting ultrasonic waves;
at least one piece of ultrasonic propagating means formed of a material having flexibility and accommodated within said insertion part, said ultrasonic propagating means having two end surfaces for entering and/or outputting ultrasonic waves, and propagating the ultrasonic waves entering from one end surface to other end surface;
guide means accommodated within said insertion part, for directing the ultrasonic waves outputted from the other end surface of said at least one piece of ultrasonic propagating means outward of said insertion part;
light applying means for applying light to an interior surface of the object; and
imaging means for obtaining image information on the interior surface of the object by detecting reflection light of the light applied to the interior surface of the object by said light applying means.

8. An apparatus to be used in optical coherence tomography for generating an image based on interference of low-coherence light and ultrasonic imaging for generating an image based on ultrasonic echoes, said apparatus comprising:
light splitting means for splitting light generated by a light source into signal light and reference light;
at least one ultrasonic transducer for generating ultrasonic waves based on a drive signal;
drive signal generating means for generating the drive signal to be supplied to said at least one ultrasonic transducer;
a probe including an insertion part to be inserted into a body of an object to be inspected and having at least one region for transmitting light and ultrasonic waves, light propagating means formed of a material having flexibility and accommodated within said insertion part, for entering the signal light split by said splitting means and propagating the signal light, at least one piece of ultrasonic propagating means formed of a material having flexibility and accommodated within said insertion part, for propagating the ultrasonic waves entering from said at least one transducer, and guide means accommodated within said insertion part, for directing the light outputted from said light propagating means outward of said insertion part and directing the ultrasonic waves outputted from said at least one piece of ultrasonic propagating means outward of said insertion part;
detecting means for detecting interference light generated by interference between the signal light reflected from the object and propagated in said light propagating means and the reference light to generate a detection signal;
first image data generating means for generating tomographic image data based on the detection signal generated by said detecting means; and
second image data generating means for generating tomographic image data based on a detection signal generated by receiving the ultrasonic waves reflected from the object.

9. An apparatus to be used in at least endoscopic imaging and ultrasonic imaging, said apparatus comprising:
at least one ultrasonic transducer for generating ultrasonic waves based on a drive signal;
drive signal generating means for generating the drive signal to be supplied to said at least one ultrasonic transducer;
a probe including an insertion part to be inserted into a body of an object to be inspected and having at least one region for transmitting ultrasonic waves, at least one piece of ultrasonic propagating means formed of a material having flexibility and accommodated within said insertion part, for propagating the ultrasonic waves entering from said at least one ultrasonic transducer, guide means accommodated within said insertion part, for directing the ultrasonic waves outputted from said at least one piece of ultrasonic propagating means outward of said insertion part, light applying means for applying light to an interior surface of the object, and imaging means for obtaining image information on the interior surface of the object by detecting reflection light of the light applied to the interior surface of the object by the light applying means;
first image data generating means for generating image data on the interior surface of the object based on the image information obtained by said imaging means; and
second image data generating means for generating tomographic image data based on a detection signal generated by receiving the ultrasonic waves reflected from the object.

10. An apparatus according to claim 8, wherein said at least one ultrasonic transducer generates the detection signal by receiving the ultrasonic waves reflected from the object and propagated in said at least one piece of ultrasonic wave propagating means.

11. An apparatus according to claim 9, wherein said at least one ultrasonic transducer generates the detection signal by receiving the ultrasonic waves reflected from the object and propagated in said at least one piece of ultrasonic wave propagating means.

12. An apparatus according to claim 8, wherein said probe further includes at least one second ultrasonic transducer accommodated within said insertion part, for generating the detection signal by receiving the ultrasonic waves reflected from the object.

13. An apparatus according to claim 9, wherein said probe further includes at least one second ultrasonic transducer accommodated within said insertion part, for generating the detection signal by receiving the ultrasonic waves reflected from the object.
